(19) United States
(12) Patent Application Publication
(10) Pub. No.: US 2021/0114968 A1
(43) Pub. Date: Apr. 22, 2021

(54) Catalytic Process For Synthesizing N,N-Dimethyl Glucamine From N Methyl Glucamine

(71) Applicant: Clariant International Ltd., Muttenz (CH)

(72) Inventors: Ertan AKGÜN, Ingolstadt (DE); Martin LINK, Mühldorf (DE); Sarah WERNER, Mühldorf (DE); Klaus RAAB, Burgkirchen (DE); Peter KLUG, Grossostheim (DE); Karl SCHEITZENEDER, Engelsberg (DE); Stefan KREUZPOINTNER, Töging (DE)

(73) Assignee: Clariant International Ltd., Muttenz (CH)

(21) Appl. No.: 16/495,360

(22) PCT Filed: Feb. 28, 2018
(86) PCT No.: PCT/EP2018/054857

§ 371 Date: Sep. 18, 2019

(30) Foreign Application Priority Data
Mar. 20, 2017 (EP) 17161762.4

(51) Int. Cl. C07C 213/08; C07C 215/10

(52) U.S. Cl. CPC C07C 213/08; C07C 215/10

(57) ABSTRACT

The invention relates to a process for synthesizing an aqueous N,N-dimethyl glucamine solution, characterized in that a formaldehyde solution is metered into an N-methyl glucamine solution in the presence of a metal catalyst at hydrogen pressure.

The N,N-dimethylglucamine (D-glucitol, 1-deoxy-1-(dimethylamino)-) described here is a valuable compound which is suitable for a host of commercial applications. These include, for example, its use as an additive in wetting agents, cleaning products, plasticizers, glidants and lubricants, and the like. There are also more recent fields of application in the manufacture of paints and inks, in crop protection, in medicine, and in cosmetics. The product, which is based on renewable raw materials, can also be used as a mild neutralizing agent or, in protonated form, as a hydrophilic cation. For this broad spectrum of applications, there are numerous quality requirements to be observed, which are not described in the processes for preparing N,N-dimethylglucamine described to date.

The process described here allows the preparation of N,N-dimethylglucamine solutions from readily commercially available N-methylglucamine under particularly mild conditions with the aid of an easily manageable single-stage process complying with the quality demands necessary for the application.

Regarding the preparation of N,N-dimethylglucamine, the literature describes a number of processes, which, however, give rise to one or more disadvantages.

EP-0614881 describes the reaction of N-monoalkylpolyhydroxy compound with aldehydes in a ratio of 1:0.9-1.5 or 1:1.1-1.03 with subsequent hydrogenation over a metallic hydrogenation catalyst, e.g., Raney-Ni, to give the tertiary dialkylpolyhydroxyamine. This reaction requires a hydrogen pressure of 10 to 150 bar and temperatures of 70 to 150° C. More particularly the reaction is carried out in two reaction steps, in which
a) first a secondary N-monoalkylpolyhydroxyamine (e.g., N-methylglucamine) is reacted with an aldehyde (e.g., formaldehyde) in a molar ratio of 1:0.90 to 1.5, preferably 1:1.03 to 1.1, in water as solvent at a temperature of 15 to 60° C., preferably of 20 to 40° C., under atmospheric pressure to give the N-monoalkylpolyhydroxyamine/aldehyde adduct, and
b) the reaction product present in step a) (and consisting substantially of water and the adduct formed) is hydrogenated in the presence of a metallic hydrogenation catalyst with hydrogen at a pressure of 10 to 150 bar, preferably 30 to 100 bar, and at a temperature of 70 to 150° C., preferably 80 to 130° C., to give the tertiary dialkylpolyhydroxyamine (e.g., N,N-dimethylglucamine).

EP-0614881 describes the possibility of achieving high yields with this process; however, no further details are described regarding the workup of the crude reaction solution. The patent specification describes the implementation of the reaction in aqueous solution, but other than the melting point there is no information on quality parameters of the dimethylglucamine. This means that there must have been a crystallization from the aqueous reaction solution, leading to the product in good yields. For the commercial use of the dimethylglucamine, however, this is not advantageous, since dimethylglucamine in solid form with a melting point of >100° C. is more difficult to handle than a liquid, and it would be advantageous to find synthesis conditions leading directly, without a purification step, to a solution of dimethylglucamine in a water-containing medium. In this case, besides yield and melting point of the recrystallized dimethylglucamine, further quality parameters are relevant, such as, for example, the color of the reaction solution and the amount of starting compounds such as N-methylglucamine, formaldehyde or byproducts such as acids which without recrystallization remain in the product and must therefore be limited. On the other hand, the process described is not advantageous on the industrial scale, since the hydrogenation, as an exothermic reaction after the combining of polyhydroxyamine and aldehyde, is not readily controllable and exhibits safety-related problems.

Although other processes describe a single-stage process, DE-2118283, for example, requires the use of Ag—Pd catalysts and likewise high temperatures of 100° C. to 250° C. The use of an expensive catalyst leads to higher production costs.

EP-A-663 389 teaches a process for preparing amino alcohols, characterized in that hydroxycarbonyl compounds are reacted with hydrogen and ammonia or with a primary or secondary amine at temperatures of 0 to 300° C. and pressures of 1 to 400 bar in the presence of catalysts whose catalytically active mass consists of 50 to 100 wt % of ruthenium.

It is known, furthermore, that in the preparation of tertiary amines from secondary amines with formaldehyde and hydrogen, the yields achievable are only poor, at below 90%. EP-0142868B1 describes the possibility of achieving better results by using supported catalysts containing a maximum of 10 wt % of Ni, Co, Ru, Rh Pd or Pt on activated carbon. Customary and favorable hydrogenation catalysts such as Raney-Ni or Raney-Co result in poorer product qualities. In this process as well, only gas-chromatographic purities were determined.

In order nevertheless to use these customary catalysts, GB-908203, for example, proposes starting the reaction from 1,3,4,6-tetraacetyl-D-glucosamine and/or using water removers such as zeolites, MgSO₄ or CaCl₂) (U.S. Pat. No. 4,190,601).

The known processes therefore afford only inadequate solutions for the preparation of N,N-dimethylglucamine solutions from N-methylglucamine. Hence the necessary quality parameters, especially the color of the reaction solution, but also the residual nickel content, can often not be maintained (see comparative examples), or the yields are too low. The known processes also lead to higher production costs, since either they require multistage syntheses, purification of the crude reaction solution, or relatively expensive starting substances (e.g., 1,3,4,6-tetraacetyl-D-glucosamine or high-purity N-methylglucamine) or expensive catalysts have to be used. Recycling of the catalysts in order to save costs is likewise not described in the known processes.

There is therefore a need for provision of an inexpensive, catalytic process for preparing N,N-dimethylglucamine solutions from inexpensive, technical or pure N-methylglucamine. For suitability for commercial fields of application such as ink and paint production, crop protection, medicine, and cosmetics, for example, the N,N-dimethylglucamine prepared by the process of the invention, and its aqueous solutions, must meet certain quality requirements in respect of color and maximum amount of defined secondary components, which cannot be achieved with the processes of the prior art.

It has surprisingly been found that in the mixing of the starting materials formaldehyde and N-methylglucamine in the presence of hydrogen, under pressure and with a catalyst, an improved product is obtained.

A subject of the invention is a process for preparing an aqueous N,N-dimethylglucamine solution, characterized in that an aqueous solution of formaldehyde is metered into an aqueous solution of N-methylglucamine in the presence of a metal catalyst at a hydrogen pressure of 10-200 bar.

A single-stage process is characterized in that all of the components are reacted simultaneously in one reactor without interim removal or purification.

The catalyst used is a nickel- or cobalt-containing catalyst, preferably a nickel-containing catalyst, especially preferably Raney nickel.

Furthermore, the reaction is to be carried out under hydrogen atmosphere and with metering of hydrogen, in order to keep the pressure constant within the specified pressure range. The concentration of the aqueous N-methylglucamine solution used is preferably in the range of 30-70 wt %, preferably 35-65 wt %, especially preferably 40-60 wt %. Formaldehyde is used as an aqueous solution, preferably with a concentration between 10 and 60 wt %, especially preferably between 30 and 40 wt %. Preferred here are formaldehyde solutions which contain a low residual methanol content.

All percentage figures are percentages by weight, unless any other percentage basis is indicated.

In the process of the invention, the molar ratio of N-methylglucamine:formaldehyde is preferably 1:1 to 1:1.5, more preferably 1:1 to 1:1.2, especially preferably 1:1.01 to 1:1.08.

The hydrogen pressure p is preferably 10-200 bar, more preferably 20-180 bar, especially preferably 70-120 bar.

The reaction temperature T in the process of the invention is preferably 30-100° C., more preferably 35-65° C., especially preferably 40-50° C.

If a dimethylglucamine solution is prepared with the process of the invention, the Hazen color number of a resulting 50% solution of N,N-dimethylglucamine in water is less than 800, preferably <400, more particularly <230.

The catalyst may be used repeatedly in the process of the invention; it is preferably reused more than five times.

Based on a 50% dimethylglucamine solution, the solutions prepared by the process of the invention contain less than 2%, preferably less than 1%, more preferably <0.25% of the initial N-methylglucamine substance.

Based on a 50% dimethylglucamine solution, the solutions prepared by the process of the invention contain preferably <0.1% of formaldehyde.

Subsequent to the hydrogenation, the process of the invention may be followed by a distillation step for removing excess water and the methanol byproduct obtained. In this case, a residual amount of preferably <0.1% of methanol is obtained.

In a further embodiment, subsequent to the process of the invention, an after-hydrogenation step is added. For this purpose, after the complete addition of the formaldehyde solution and the end of hydrogen absorption, a hydrogenation is performed at 60-110° C. The product obtained from the process of the invention is preferably not isolated before the after-hydrogenation.

The product produced by the process of the invention yields the following quality parameters (based on a 50% solution of N,N-dimethylglucamine in water):
The products have a Hazen color number of less than 800, preferably less than 400; more particularly, of less than 230. This is decisive, for example, for the use in cosmetics or in paints and inks.
The residual N-methylglucamine content (determined by GC) is less than 2 wt %, preferably less than 1 wt %, more particularly less than 0.25 wt %. Elevated values would lead to a higher fraction of secondary amines and to potential nitrosatability, which is disruptive to a large number of applications and formulations.
Furthermore, the presence of short-chain free and also chemically bonded monocarboxylic acids may be a disruption to various applications, as a result, for example, of salt formation. As a lead substance, a description is given here of the formic acid content (determined via ion chromatography), which with the process of the invention is below 1 wt %, preferably below 0.5 wt %, more particularly below 0.1 wt %.
The content of free and chemically bonded formaldehyde with the process described is below 0.5 wt %, preferably below 0.1 wt %, more particularly below 0.05 wt %.
The process of the invention furnishes product which contains low levels of unwanted metallic impurities such as aluminum, cobalt or nickel, for example (determined via ICP-OES). The nickel content is less than 50 ppm, preferably less than 20 ppm, more particularly less than 10 ppm.
Likewise disruptive to the application are volatile organic compounds such as, for example, methanol, the amount of which by the process described is below 0.7 wt %, preferably below 0.5 wt %, especially preferably below 0.1 wt %.

The process of the invention is carried out preferably in a stirred reactor or in a loop reactor with an external pumped circulation and mixing. This reactor is temperature-controlled in order to be able to intercept any exothermic or endothermic temperature changes occurring, and to keep the temperature constant throughout the reaction time. During the reaction, formaldehyde is metered in continuously or in sections, preferably continuously, and is carried out with or without further reaction time without further addition of formaldehyde. The precise reaction time can be determined via samples taken and in process controls.

The stated catalysts (e.g., Raney-Ni) remain in the reactor after filtration and are used for further syntheses. This affords a critical advantage over other processes, with which the catalyst cannot be reused or can be reused only a few times, with a considerable increase in the production costs. Any losses of catalyst material due to filtration that do occur can be made up before each new batch.

In order to remove any volatile components present in the reaction solution or to set the desired concentration of water, the reaction solution obtained can be worked up by stripping or distillation or a similar method known to the skilled person. Stripping may take place with addition of nitrogen or water at temperatures between 20-100° C., preferably 30-80° C., especially preferably between 40 and 60° C., under the corresponding vapor pressure of water.

The process of the invention therefore leads to mixtures of N-methylglucamine and water in a proportion, for example, of 1:99 to 99:1, preferably 30:70 to 90:10, especially preferably 45:55 to 80:20.

If even lower concentrations of metals are desired for the use of the product, workup may take place over an ion exchanger or by a similar method known to the skilled person.

Furthermore, the N,N-dimethylglucamine solution prepared by the process of the invention may also be prepared in the form of pure, crystalline N,N-dimethylglucamine. This may be done by workup methods known to the skilled person, examples being distillative removal of the water and/or recrystallization from various solvents such as, for instance, alcohol or alcohol/water mixtures, and is important for use in the pharmaceutical and medical sectors.

For the process of the invention, paraformaldehyde or aqueous formaldehyde solutions are used. Preferred are aqueous formaldehyde solutions with an active content of 30-40%, especially preferably those containing a low methanol content.

The N-methylglucamine used can be employed in the form of high-purity recrystallized product, but preferably in technical quality. Technical N-methylglucamine can in principle be prepared from glucose syrup according to WO-92/06073 and without further purification is obtained as an aqueous solution at approximately 60%.

This may be purified further by recrystallization. For N,N-dimethylglucamine with good color, however, recrystallization is unnecessary.

The process of the invention allows N,N-dimethylglucamine to be produced in an inexpensive and simple process. In comparison to existing processes (e.g., EP-614881A1), the process is easier to control and leads to lower production costs, better product quality, and therefore to a broader spectrum of usage.

The reason for the low production costs is that the single-stage operation does not require an intermediate to be isolated. In comparison to EP-614881A1, isolation of the adduct before the hydrogenation is not necessary and therefore the plant occupation time is minimized. The inexpensive nickel-containing or cobalt-containing catalyst used likewise leads to a reduction in the costs by comparison with other processes, which require the use of costly catalysts such as Ru, Ag—Pd or Pt. The production costs are likewise lowered considerably through the possibility of reusing the catalyst for numerous reaction cycles.

Surprisingly, in contrast to U.S. Pat. No. 4,190,601, it has been found that the presence of water does not disrupt the reaction. Water can therefore be used as solvent. When the described, suitable concentrations are observed, this leads to numerous advantages in connection with handling, such as, for example, a lowering of the viscosity and the prevention of crystallization of the product at room temperature and/or reaction temperature. Using water as a solvent also reduces the costs, lowers the potential for hazard, and avoids toxic/hazardous wastes.

Observing the quality parameters necessary to the application is not adequately described in the case of the known processes described in the prior art, and/or the processes result in relatively poor quality. The comparative examples show that the process of the invention may have advantages in terms of the quality parameters, especially the color of product, and hence that costly purification can be avoided.

### EXAMPLES

| | | | Hydro- | Gardner | Hazen | | | |
|---|---|---|---|---|---|---|---|---|
| | | | genation | color at | color at | | | |
| | | Hydro- | temp- | 50% | 50% | | | Form- |
| | 1-stage/ | genation | erature | active | active | NMG | Ni | aldehyde |
| | 2-stage | pressure | [° C.] | content | content | [%] | [ppm] | [%] |
| 1 (comp.) | 2-stage | 30 | 100 | 9.4 | n.m. | 0.91 | 35 | 0.03 |
| 2 (comp.) | 2-stage | 100 | 130 | 16.9 | n.m. | 6.0 | 29 | 0.01 |
| 1 | 1-stage | 28 | 60 | 2.5 | 350 | 0.06 | 2 | 0.02 |
| 2 | 1-stage, | 28 | 60 | 1.1 | 200 | 0.08 | 2 | 0.02 |
| | repeat | | | | | | | |
| 3 | 1-stage | 100 | 60 | 0.6 | 105 | 0.10 | 2 | 0.02 |
| 4 | 1-stage, | 100 | 60 | 0.6 | 115 | 0.05 | 3 | 0.01 |
| | repeat | | | | | | | |
| 3 (comp.) | 1-stage | 3 | 60 | 9 | n.m. | 0.17 | 62 | 0.03 |
| 4 (comp.) | 1-stage | 3 | 60 | 8 | n.m. | 0.9 | 45 | 0.03 |
| 5 (comp.) | 1-stage | 85 | 40 | 0.5 | 81 | 0.13 | 0.6 | 0.01 |

n.m. = not measurable

Gardner color number and Hazen color number:

The clear, aqueous dimethylglucamine solutions without any gas bubbles were introduced into 10 mm rectangular cuvettes. The color numbers were measured at room temperature in a LICO 690 colorimeter from Hach Lange.

Total amine number by acid-base titration:

The samples, weighed out accurately on an analytical balance, were dissolved in glacial acetic acid and titrated with 0.1 molar perchloric acid in glacial acetic acid, using a titroprocessor from Metrohm.

Solids content (105° C./2 hours):

The samples, weighed out precisely, were dried to constant weight in a drying cabinet at 105° C. for two hours by evaporation of the water, and after drying were precisely weighed again.

Water (Karl Fischer titration):

The water content of the precisely weighed-out samples was determined by the Karl Fischer titration method using Karl Fischer solvent and Karl Fischer titrant.

N-methylglucamine, N,N-dimethylglucamine and sorbitol (GC):

The samples were completely acetylated with a very large excess of acetic anhydride/pyridine at 80° C. The N-methylglucamine, N,N-dimethylglucamine and sorbitol contents were determined by gas chromatography on a 60 m Agilent HP-5 column, using decanol as internal standard and using an FID detector.

Methanol (GC):

The methanol content was determined by a gas-chromatographic method for volatile substances, using isobutanol as internal standard and using a TCD detector.

Formaldehyde (free and chemically bonded):

To release the chemically bonded formaldehyde, the samples were heated with aqueous sulfuric acid in the presence of 2,4-dinitrophenylhydrazine. Through the reaction of the formaldehyde with 2,4-dinitrophenylhydrazine, 2,4-dinitrophenylhydrazone was formed. The 2,4-dinitrophenylhydrazone content was subsequently determined by HPLC liquid chromatography. The free formaldehyde and chemically bonded formaldehyde content was captured as a sum total.

Nitrosamines (total NNO):

The samples were analyzed for total nitrosamine content in a method based on the ATNC (apparent total nitrosamine content) method. With chemical denitrosation in an acidic medium, nitrogen monoxide was released from the nitrosamines and was subsequently determined quantitatively using a very sensitive detector specific for nitrogen monoxide. Calculation and indication of content were made in the form of the NNO content (>N—N═O with molar mass 44 g/mol).

Nickel (ICP-OES):

The nickel content of the samples was determined by ICP-OES (inductively coupled plasma optical emission spectrometry) in a method based on DIN EN ISO 11885.

Formic acid (free and chemically bonded, IC):

The samples were pretreated with heating in an aqueous-alkaline medium in order by hydrolysis to liberate the formic acid, bonded chemically in the form of ester and amide. The amount of formic acid or salts thereof already present before the alkaline hydrolysis, and of formic acid and/or salts thereof liberated only by the alkaline hydrolysis, was determined as a sum total by ion chromatography (IC).

### Comparative Example 1: Example 2 from EP-0614881

In a 2 liter round-bottom glass flask equipped with stirrer, thermometer and electrical heating under atmospheric pressure, 1500 g of a 43%, aqueous N-methylglucamine solution were produced by diluting a 60% N-methylglucamine solution (Gardner color number 1.7, Hazen color number 263). The 43%, aqueous N-methylglucamine solution was heated to 35° C. with stirring. From a dropping funnel, with further stirring, a total of 288.7 g of a 36.5%, aqueous formaldehyde solution were added dropwise over the course of half an hour at 35° C. The reaction to give the N-methylglucamine-formaldehyde adduct was slightly exothermic. The Gardner color number of the pale-yellow, clear, aqueous N-methylglucamine-formaldehyde adduct solution was 0.8 and the Hazen color number was 151.

Immediately after the end of the dropwise addition, a portion of the aqueous N-methylglucamine-formaldehyde adduct solution amounting to 1300 g was taken from the 2 liter round-bottom glass flask and was introduced at room temperature into a 2 liter stirred autoclave.

The 2 liter stirred autoclave was equipped with stirrer, heating, cooling, supply lines for hydrogen and nitrogen, temperature measurement, pressure measurement, and safety valve. 20.4 g of Raney nickel were introduced under nitrogen into the 2 liter stirred autoclave.

The 2 liter stirred autoclave was closed. Three times, 10 bar of nitrogen were injected, with depressurization each time. Thereafter, three times, 10 bar of hydrogen were injected, with depressurization each time. After the leak test and depressurization, the stirring speed was set to 800 rpm. With further stirring, heating took place to 100° C. and then hydrogen was supplied. The exothermic hydrogenation took place, with renewed injection of the hydrogen consumed, at 800 rpm, 100° C. and a hydrogen pressure of 30 bar. After the end of the evident absorption of hydrogen, stirring was continued for two hours at 100° C. and a hydrogen pressure of 30 bar. This was followed by cooling to 30° C., depressurization, purging with nitrogen, and the emptying of the 2 liter stirred autoclave. The Raney nickel catalyst was separated off by a pressure filtration under nitrogen. The liquid filtrate was dark brown and had a Gardner color number of 8.

This dark brown filtrate was subjected to initial distillation in a rotary evaporator at 60° C. under a pressure of 20 mbar. The distillate contained primarily water, a little methanol, and traces of other low boilers. The viscous N,N-dimethylglucamine having undergone initial distillation was adjusted to an active ingredient content of 51% and DI water content of 49% by addition of DI water with thorough mixing at 60° C. This product solution was subjected to analysis. Apart from the losses involved in emptying the autoclave and working up the solution, the yield of dissolved N,N-dimethylglucamine in the DMG 50 was virtually quantitative.

The product obtained was characterized as follows:

| Appearance at 20° C.: | clear, liquid, dark brown | |
|---|---|---|
| Gardner color number: | 9.4 | |
| Hazen color number: | not measurable, since too dark | |
| Total amine number (titration): | 133 | mg KOH/g |
| Solids content (105° C./2 hours): | 51.4% | (m/m) |
| Water (Karl-Fischer titration): | 49% | (m/m) |
| N-Methylglucamine (GC): | 0.91% | (m/m) |
| N,N-Dimethylglucamine (GC): | 44.5% | (m/m) |
| Sorbitol (GC): | 0.6% | (m/m) |
| Methanol (GC): | <0.1% | (m/m) |
| Formaldehyde (free and chemically | 0.026% | (m/m) |
| bonded, LC): | | |
| Nitrosamines (total NNO): | <50 | μg/kg |
| Nickel (ICP-OES): | 35 | μg/g |
| Formic acid (free and chemically | 0.70% | (m/m) |
| bonded) | | |

### Comparative Example 2: As Per Example 3 from EP-0614881

In a 2 liter round-bottom glass flask equipped with stirrer, thermometer and electrical heating under atmospheric pressure, 1500 g of a 43%, aqueous N-methylglucamine solution were produced by diluting a 60%, technical N-methylglucamine solution (Gardner color number 1.7, Hazen color number 263) with DI water.

The 43%, aqueous N-methylglucamine solution was heated to 35° C. with stirring. From a dropping funnel, with further stirring, a total of 288.7 g of a 36.5%, aqueous formaldehyde solution were added dropwise over the course of half an hour at 35° C. The reaction to give the N-methylglucamine-formaldehyde adduct was slightly exothermic. Thereafter the clear, pale yellow reaction mixture was additionally stirred further for an hour at 35° C. The Gardner color number of this pale-yellow, aqueous N-methylglucamine-formaldehyde adduct solution was 0.8 and the Hazen color number was 162.

Thereafter, a portion of the aqueous N-methylglucamine-formaldehyde adduct solution amounting to 1300 g was taken from the 2 liter round-bottom glass flask and was introduced at room temperature into a 2 liter stirred autoclave.

The 2 liter stirred autoclave was equipped with stirrer, heating, cooling, supply lines for hydrogen and nitrogen, temperature measurement, pressure measurement, and safety valve. 20.4 g of Raney nickel were introduced under nitrogen into the 2 liter stirred autoclave.

The 2 liter stirred autoclave was closed. Three times, 10 bar of nitrogen were injected, with depressurization each time. Thereafter, three times, 10 bar of hydrogen were injected, with depressurization each time. After the leak test and depressurization, the stirring speed was set to 800 rpm. With further stirring, heating took place to 125° C. and then hydrogen was supplied. The exothermic hydrogenation took place, with renewed injection of the hydrogen consumed, at 800 rpm, 130° C. and a hydrogen pressure of 100 bar. After the end of the evident absorption of hydrogen, stirring was continued for two hours at 130° C. and a hydrogen pressure of 100 bar. This was followed by cooling to 30° C., depressurization, purging with nitrogen, and the emptying of the 2 liter stirred autoclave. The Raney nickel catalyst was separated off by a pressure filtration under nitrogen. The liquid filtrate was deeply dark brown and had a Gardner color number of 13.4.

This deeply dark brown filtrate was subjected to initial distillation in a rotary evaporator at 60° C. under a pressure of 20 mbar. The distillate contained primarily water, a little methanol, and traces of other low boilers. The viscous N,N-dimethylglucamine having undergone initial distillation was adjusted to an active ingredient content of 51% and DI water content of 49% by addition of DI water with thorough mixing at 60° C. The resulting product solution was subjected to analysis. Apart from the losses involved in emptying the autoclave and working up the solution, the yield of dissolved N,N-dimethylglucamine in the DMG 50 was virtually quantitative.

The product obtained was characterized as follows:

| Appearance at 20° C.: | clear, liquid, deeply dark brown | |
|---|---|---|
| Gardner color number: | 16.9 | |
| Hazen color number: | not measurable, since too dark | |
| Total amine number (titration): | 133 | mg KOH/g |
| Solids content (105° C./2 hours): | 52.6% | (m/m) |
| Water (Karl-Fischer titration): | 49% | (m/m) |
| N-Methylglucamine (GC): 6.0% (m/m) | | |
| N,N-Dimethylglucamine (GC): | 35.9??% | (m/m) |
| Sorbitol (GC): | 0.6% | (m/m) |
| Methanol (GC): | <0.1% | (m/m) |
| Formaldehyde (free and chemically | 0.012% | (m/m) |
| bonded, LC): | | |
| Nitrosamines (total NNO): | <50 | μg/kg |
| Nickel (ICP-OES): | 29 | μg/g |
| Formic acid (free and chemically | 0.90% | (m/m) |
| bonded) | | |

### Example 1

A 10 liter stirred autoclave equipped with temperature measurement, pressure measurement, safety valve, gas-introduction stirrer and immersed tube was charged under a countercurrent of nitrogen with 4.95 kg of 57.3% N-methylglucamine solution in water at 60° C., Gardner color number: 0.9, Hazen color number: 158, and 200 g of water-moist Raney nickel (containing 40% water). Rinsing took place with 200 g of DI water. The 10 liter stirred autoclave was closed. Three times, 10 bar of nitrogen were injected, with depressurization each time. Thereafter, three times, 10 bar of hydrogen were injected, with depressurization each time. After the leak test, the stirring speed was set to 1100 rpm, the internal temperature to 60° C. and the hydrogen pressure to 28 bar. Over the course of 4 hours and 10 minutes, 1.245 kg of aqueous formaldehyde solution (36.8% formaldehyde dissolved in water with methanol) were metered continuously, using a pump, into the 10 liter stirred autoclave. The molar ratio of N-methylglucamine to the total formaldehyde after the end of formaldehyde metering was 1.00:1.05. The stirring speed was held at 1100 rpm and the internal temperature at 59-64° C. The hydrogen consumed was replaced by supplying fresh hydrogen and in this way the hydrogen pressure in the 10 liter stirred autoclave was held at 25-28 bar. After the end of absorption of hydrogen, stirring was continued for 3 hours at 60° C. and 28 bar, followed by heating to 90° C. and stirring for a further 3 hours at 90° C. and 28 bar. After that, cooling took place to 30° C. and the stirrer was switched off. For an hour, the Raney nickel settled. The major part of the crude, aqueous N,N-dimethylglucamine was slowly pressed out of the 10 liter stirred autoclave, via an immersed tube reaching not quite to the base, and was separated from small fractions of the suspended Raney nickel by means of pressure filtration. The major part of the Raney nickel remained, with a small residue of aqueous N,N-dimethylglucamine, beneath the immersed tube in the 10 liter stirred autoclave, for the next batch.

The filtered, aqueous N,N-dimethylglucamine was subjected to initial distillation in a rotary evaporator at 60° C. under a pressure of 20 mbar. The distillate contained primarily water, a little methanol, and traces of other low boilers. The viscous N,N-dimethylglucamine having undergone initial distillation was adjusted to an active ingredient content of 50% and DI water content of 50% by addition of DI water with thorough mixing at 60° C. Apart from the amount remaining under the immersed tube in the 10 liter stirred autoclave and the samples taken, the yield of dissolved N,N-dimethylglucamine was virtually quantitative.

The product obtained was characterized as follows:

| Appearance | clear, liquid, yellow | |
|---|---|---|
| Gardner color number: | 2.5 | |
| Hazen color number: | 350 | |
| Total amine number (titration): | 129 | mg KOH/g |
| Solids content (105° C./2 hours): | 50.0% | (m/m) |
| Water (Karl-Fischer titration): | 50% | (m/m) |
| N-Methylglucamine (GC): | 0.06% | (m/m) |
| N,N-Dimethylglucamine (GC): | 43.5% | (m/m) |
| Sorbitol (GC): | 1.0% | (m/m) |
| Methanol (GC): | <0.01% | (m/m) |
| Formaldehyde (free and chemically | 0.015% | (m/m) |
| bonded, LC): | | |
| Nitrosamines (total NNO): | <50 | μg/kg |
| Nickel (ICP-OES): | 2 | μg/g |
| Formic acid (free and chemically | 0.024% | (m/m) |
| bonded) (ion chromatography after | | |
| alkaline hydrolysis, IC) | | |

### Example 2

The 10 liter stirred autoclave with aqueous N,N-dimethylglucamine and used Raney nickel beneath the immersed tube from the previous batch (example 1) was charged under a countercurrent of nitrogen with 4.95 kg of liquid, 57.3% N-methylglucamine solution in water at 60° C. (Gardner color number: 0.9, Hazen color number: 158) and 30 g of fresh, water-moist Raney nickel (containing 40% water). Rinsing was carried out with 200 g of DI water. The 10 liter stirred autoclave was closed. Three times, 10 bar of nitrogen were injected, with depressurization each time. Thereafter, three times, 10 bar of hydrogen were injected, with depressurization each time. After the leak test, the stirring speed was set to 1100 rpm, the internal temperature to 60° C. and the hydrogen pressure to 28 bar. Over the course of 3 hours and 50 minutes, 1.244 kg of aqueous formaldehyde (36.8% formaldehyde dissolved in water with methanol) were metered continuously, using a pump, into the 10 liter stirred autoclave. The molar ratio of N-methylglucamine to the total formaldehyde after the end of the formaldehyde metering was 1.00:1.05. The stirring speed was held at 1100 rpm and the internal temperature at 55-64° C. The hydrogen consumed was replaced by a supply of fresh hydrogen and in this way the hydrogen pressure in the 10 liter stirred autoclave was held at 25-28 bar. The continuous formation of the N-methylglucamine/formaldehyde adduct and the continuous catalytic hydrogenation of this product to form N,N-dimethylglucamine took place simultaneously. After the end of absorption of hydrogen, stirring was continued for 3 hours at 60° C. and 28 bar, followed by heating to 90° C. and stirring for a further 3 hours at 90° C. and 28 bar. This was followed by cooling to 30° C., and the stirrer was switched off. The Raney nickel settled for an hour. Via an immersed tube reaching not quite to the base, the major part of the crude, aqueous N,N-dimethylglucamine was pressed out slowly from the 10 liter stirred autoclave and separated from small fractions of the suspended Raney nickel by means of pressure filtration. The major part of the Raney nickel remained, with a small residue of aqueous N,N-dimethylglucamine, beneath the immersed tube in the 10 liter stirred autoclave, for the next batch.

The filtered, aqueous N,N-dimethylglucamine was subjected to initial distillation in a rotary evaporator at 60° C. under a pressure of 20 mbar. The distillate contained primarily water, a little methanol, and traces of other low boilers. The viscous N,N-dimethylglucamine having undergone initial distillation was adjusted to an active ingredient content of 50% and DI water content of 50% by addition of DI water with thorough mixing at 60° C. Apart from the samples taken, the yield of dissolved N,N-dimethylglucamine was virtually quantitative.

The product obtained was characterized as follows:

| Appearance | clear, liquid, yellow | |
|---|---|---|
| Gardner color number: | 1.1 | |
| Hazen color number: | 200 | |
| Total amine number (titration): | 139 | mg KOH/g |
| Solids content (105° C./2 hours): | 50.1% | (m/m) |
| Water (Karl-Fischer titration): | 49.8% | (m/m) |
| N-Methylglucamine (GC)*: | 0.08% | (m/m) |
| N,N-Dimethylglucamine (GC): | 46.7% | (m/m) |
| Sorbitol (GC): | 1.1% | (m/m) |
| Methanol (GC): | <0.01% | (m/m) |
| Formaldehyde (free and chemically | 0.021% | (m/m) |
| bonded, LC): | | |
| Nitrosamines (total NNO): | <50 | μg/kg |
| Nickel (ICP-OES): | 2 | μg/g |

Formic acid (free and chemically
bonded) (ion chromatography after
alkaline hydrolysis, IC)

### Example 3

A 10 liter stirred autoclave equipped with temperature measurement, pressure measurement, safety valve, gas-introduction stirrer and immersed tube was charged under a countercurrent of nitrogen with 4.97 kg of liquid, 57.3% N-methylglucamine solution in water at 60° C. (Gardner color number: 0.9, Hazen color number: 158) and with 200 g of water-moist Raney nickel (containing 40% water). Rinsing was carried out with 200 g of DI water. The 10 liter stirred autoclave was closed. Three times, 10 bar of nitrogen were injected, with depressurization each time. Thereafter, three times, 10 bar of hydrogen were injected, with depressurization each time. After the leak test, the stirring speed was set to 1100 rpm, the internal temperature to 60° C. and the hydrogen pressure to 90 bar. Over the course of 3 hours and 15 minutes, 1.249 kg of aqueous formaldehyde (36.8% formaldehyde dissolved in water with methanol) were metered continuously, using a pump, into the 10 liter stirred autoclave. The molar ratio of N-methylglucamine to the total formaldehyde after the end of formaldehyde metering was 1.00:1.05. The stirring speed was held at 1100 rpm and the internal temperature at 55-61° C. The hydrogen consumed was replaced by a supply of fresh hydrogen, and in this way the hydrogen pressure in the 10 liter stirred autoclave was held at 97-101 bar. The continuous formation of the N-methylglucamine/formaldehyde adduct and the continuous catalytic hydrogenation of said product to form N,N-dimethylglucamine took place simultaneously. The conversion was monitored by samples taken and analysis of said samples by GC, NMR, and titration for determining the amine number. After the end of absorption of hydrogen, stirring was continued for 3 hours at 60° C. and 100 bar, followed by heating to 90° C. and stirring for a further 3 hours at 90° C. and 105 bar. After that, cooling took place to 30° C. and the stirrer was switched off. The Raney nickel settled for an hour. Via an immersed tube reaching not quite to the base, the major part of the crude aqueous N,N-dimethylglucamine was pressed out slowly from the 10 liter stirred autoclave and was separated from small fractions of the suspended Raney nickel by means of pressure filtration. The major part of the Raney nickel remained, with a small residue of aqueous N,N-dimethylglucamine, beneath the immersed tube in the 10 liter stirred autoclave, for the next batch.

The filtered, aqueous N,N-dimethylglucamine was subjected to initial distillation in a rotary evaporator at 60° C. under a pressure of 20 mbar. The distillate contained primarily water, a little methanol, and traces of other low boilers. The viscous N,N-dimethylglucamine having undergone initial distillation was adjusted to an active ingredient content of 50% and DI water content of 50% by addition of DI water with thorough mixing at 60° C. Apart from the amount remaining under the immersed tube in the 10 liter stirred autoclave and the samples taken, the yield of dissolved N,N-dimethylglucamine was virtually quantitative.

The product obtained was characterized as follows:

| Appearance | clear, liquid, pale yellow | |
|---|---|---|
| Gardner color number: | 0.6 | |
| Hazen color number: | 105 | |
| Total amine number (titration): | 133.5 | mg KOH/g |
| Solids content (105° C./2 hours): | 50.2 | |
| Water (Karl-Fischer titration): | 49.6% | (m/m) |
| N-Methylglucamine (GC) | 0.10% | (m/m) |
| N,N-Dimethylglucamine (GC): | 43.4% | (m/m) |
| Sorbitol (GC): | 1.1% | (m/m) |
| Methanol (GC): | <0.01% | (m/m) |
| Formaldehyde (free and chemically | 0.024% | (m/m) |
| bonded, LC): | | |
| Nitrosamines (total NNO): | <50 | μg/kg |
| Nickel (ICP-OES): | 2 | μg/g |

| | |
|---|---|
| Formic acid (free and chemically | 0.015% (m/m), depending on |
| bonded) (ion chromatography after | the NMG grade |
| alkaline hydrolysis, IC) | |

### Example 4

The 10 liter stirred autoclave with aqueous N,N-dimethylglucamine and used Raney nickel beneath the immersed tube from the previous batch (example 3) was charged under a countercurrent of nitrogen with 4.98 kg of liquid, 57.3% N-methylglucamine solution in water at 60° C. (Gardner color number: 0.9, Hazen color number: 158) and 30 g of fresh, water-moist Raney nickel (containing 40% water). Rinsing was carried out with 200 g of DI water. The 10 liter stirred autoclave was closed. 10 bar of nitrogen was injected three times, with depressurization after each injection. After that, 10 bar of hydrogen was injected three times, with depressurization after each injection. Following the leak test, the stirring speed was set to 1100 rpm, the internal temperature to 60° C., and the hydrogen pressure to 90 bar. Over the course of 3 hours and 50 minutes, 1.252 kg of aqueous formaldehyde (36.8% formaldehyde dissolved in water with methanol) were metered continuously, using a pump, into the 10 liter stirred autoclave. The molar ratio of N-methylglucamine to the total formaldehyde after the end of the formaldehyde metering was 1.00:1.05. The stirring speed was held at 1100 rpm and the internal temperature at 57-62° C. The hydrogen consumed was replaced by a supply of fresh hydrogen and in this way the hydrogen pressure in the 10 liter stirred autoclave was held at 96-99 bar. The continuous formation of the N-methylglucamine/formaldehyde adduct and the continuous catalytic hydrogenation of this product to form N,N-dimethylglucamine took place simultaneously. By taking samples and analyzing these samples by GC, NMR and titration for determining the amine number, the conversion was monitored. After the end of absorption of hydrogen, stirring was continued for 3 hours at 60° C. and 100 bar, followed by heating to 90° C. and stirring for a further 3 hours at 90° C. and 108 bar. This was followed by cooling to 30° C., and the stirrer was switched off. The Raney nickel settled for an hour. Via an immersed tube reaching not quite to the base, the major part of the crude, aqueous N,N-dimethylglucamine was pressed out slowly from the 10 liter stirred autoclave and separated from small fractions of the suspended Raney nickel by means of pressure filtration. The major part of the Raney nickel remained, with a small residue of aqueous N,N-dimethylglucamine, beneath the immersed tube in the 10 liter stirred autoclave, for the next batch.

The filtered, aqueous N,N-dimethylglucamine was subjected to initial distillation in a rotary evaporator at 60° C. under a pressure of 20 mbar. The distillate contained primarily water, a little methanol, and traces of other low boilers. The viscous N,N-dimethylglucamine having undergone initial distillation was adjusted to an active ingredient content of 50% and DI water content of 50% by addition of DI water with thorough mixing at 60° C. Apart from the samples taken, the yield of dissolved N,N-dimethylglucamine was virtually quantitative.

The product obtained was characterized as follows:

| Appearance: | clear, liquid, pale-yellow | |
|---|---|---|
| Gardner color number: | 0.6 | |
| Hazen color number: | 115 | |
| Total amine number (titration): | 132 | mg KOH/g |
| Solids content (105° C./2 hours): | 49.9% | |
| Water (Karl-Fischer titration): | 50.3% | (m/m) |
| N-Methylglucamine (GC)*: | 0.05% | (m/m) |
| N,N-Dimethylglucamine (GC): | 45.0% | (m/m) |
| Sorbitol (GC): | 1.0% | (m/m) |
| Methanol (GC): | 0.01% | (m/m) |
| Formaldehyde (free and chemically | 0.012% | (m/m) |
| bonded, LC): | | |

| | |
|---|---|
| Nitrosamines (total NNO): | <50 μg/kg, depending on |
| | the NMG grade |
| Nickel (ICP-OES): | 2 μg/g, depending on |
| | the NMG grade |
| Formic acid (free and chemically | 0.024% (m/m), depending on |
| bonded) (ion chromatography after | the NMG grade |
| alkaline hydrolysis, IC) | |

### Comparative Example 3 (Low Hydrogen Pressure)

A 10 liter stirred autoclave equipped with temperature measurement, pressure measurement, safety valve, gas-introduction stirrer and immersed tube was charged under a countercurrent of nitrogen with 5.024 kg of liquid, 57.3% N-methylglucamine solution in water at 60° C. (Gardner color number: 0.9, Hazen color number: 158) and 200 g of water-moist Raney nickel (containing 40% water). Rinsing was carried out with 200 g of DI water. The 10 liter stirred autoclave was closed. 10 bar of nitrogen was injected three times, with depressurization after each injection. After that, 10 bar of hydrogen was injected three times, with depressurization after each injection. Following the leak test, the stirring speed was set to 1100 rpm, the internal temperature to 60° C., and the hydrogen pressure to 3 bar absolute. Over the course of 2 hours and 45 minutes, 1.263 kg of aqueous formaldehyde (36.8% formaldehyde dissolved in water with methanol) were metered continuously, using a pump, into the 10 liter stirred autoclave. The molar ratio of N-methylglucamine to the total formaldehyde after the end of the formaldehyde metering was 1.00:1.05. The stirring speed was held at 1100 rpm and the internal temperature at 54-63° C. The hydrogen consumed was replaced by a supply of fresh hydrogen and in this way the hydrogen pressure in the 10 liter stirred autoclave was held at 2.9-3.2 bar absolute. The continuous formation of the N-methylglucamine/formaldehyde adduct and the continuous catalytic hydrogenation of this product to form N,N-dimethylglucamine took place simultaneously. The conversion was monitored by taking samples and analyzing these samples by GC, NMR and titration for determining the amine number. After the end of absorption of hydrogen, stirring was continued for 3 hours at 60° C. and 3.5 bar absolute, followed by heating to 90° C. and stirring for a further 3 hours at 90° C. and 3.9 bar absolute. This was followed by cooling to 30° C., and the stirrer was switched off. The Raney nickel settled for an hour. Via an immersed tube reaching not quite to the base, the major part of the crude, aqueous N,N-dimethylglucamine was pressed out slowly from the 10 liter stirred autoclave and separated from small fractions of the suspended Raney nickel by means of pressure filtration. The major part of the Raney nickel remained, with a small residue of aqueous N,N-dimethylglucamine, beneath the immersed tube in the 10 liter stirred autoclave, for the next batch.

The filtered, aqueous N,N-dimethylglucamine was subjected to initial distillation in a rotary evaporator at 60° C. under a pressure of 20 mbar. The distillate contained primarily water, a little methanol, and traces of other low boilers. The viscous N,N-dimethylglucamine having undergone initial distillation was adjusted to an active ingredient content of 50% and DI water content of 50% by addition of DI water with thorough mixing at 60° C. Apart from the amount remaining beneath the immersed tube in the 10 liter stirred autoclave and the samples taken, the yield of dissolved N,N-dimethylglucamine was virtually quantitative.

The product obtained was characterized as follows:

| Appearance: | clear, liquid, brown | |
|---|---|---|
| Gardner color number: | 9 | |
| Hazen color number: | >1000 | |
| Total amine number (titration): | 3.54 | meq/g |
| Solids content (105° C./2 hours): | 50.3 | |
| Water (Karl-Fischer titration): | 49.8% | (m/m) |
| N-Methylglucamine (GC): | 0.17% | (m/m) |
| N,N-Dimethylglucamine (GC): | 44.3% | (m/m) |
| Sorbitol (GC): | 0.8% | (m/m) |
| Methanol (GC): | <0.01% | (m/m) |
| Formaldehyde (free and chemically | 0.029% | (m/m) |
| bonded, LC): | | |
| Nitrosamines (total NNO): | <50 | μg/kg |
| Nickel (ICP-OES): | 62 | μg/g |
| Formic acid (free and chemically | 0.15% | (m/m) |
| bonded) (ion chromatography after | | |
| alkaline hydrolysis, IC) | | |

### Comparative Example 4 (Low Hydrogen Pressure)

The 10 liter stirred autoclave with aqueous N,N-dimethylglucamine and used Raney nickel beneath the immersed tube from the preceding batch (example 5) was charged under a countercurrent of nitrogen with 5.026 kg of liquid, 57.3% N-methylglucamine solution in water at 60° C. (Gardner color number: 0.9, Hazen color number: 158) and 30 g of fresh, water-moist Raney nickel (containing 40% water). Rinsing was carried out with 200 g of DI water. The 10 liter stirred autoclave was closed. 10 bar of nitrogen was injected three times, with depressurization after each injection. After that, 10 bar of hydrogen was injected three times, with depressurization after each injection. Following the leak test, the stirring speed was set to 1100 rpm, the internal temperature to 60° C., and the hydrogen pressure to 3 bar absolute. Over the course of 3 hours and 30 minutes, 1.264 kg of aqueous formaldehyde (36.8% formaldehyde dissolved in water with methanol) were metered continuously, using a pump, into the 10 liter stirred autoclave. The molar ratio of N-methylglucamine to the total formaldehyde after the end of the formaldehyde metering was 1.00:1.05. The stirring speed was held at 1100 rpm and the internal temperature at 56-63° C. The hydrogen consumed was replaced by a supply of fresh hydrogen and in this way the hydrogen pressure in the 10 liter stirred autoclave was held at 3.5-3.9 bar absolute. The continuous formation of the N-methylglucamine/formaldehyde adduct and the continuous catalytic hydrogenation of this product to form N,N-dimethylglucamine took place simultaneously. The conversion was monitored by taking samples and analyzing these samples by GC, NMR and titration for determining the amine number. After the end of absorption of hydrogen, stirring was continued for 3 hours at 60° C. and 3.9 bar absolute, followed by heating to 90° C. and stirring for a further 3 hours at 90° C. and 3.8 bar absolute. This was followed by cooling to 30° C., and the stirrer was switched off. The Raney nickel settled for an hour. Via an immersed tube reaching not quite to the base, the major part of the crude, aqueous N,N-dimethylglucamine was pressed out slowly from the 10 liter stirred autoclave and separated from small fractions of the suspended Raney nickel by means of pressure filtration. The major part of the Raney nickel remained, with a small residue of aqueous N,N-dimethylglucamine, beneath the immersed tube in the 10 liter stirred autoclave, for the next batch.

The filtered, aqueous N,N-dimethylglucamine was subjected to initial distillation in a rotary evaporator at 60° C. under a pressure of 20 mbar. The distillate contained primarily water, a little methanol, and traces of other low boilers. The viscous N,N-dimethylglucamine having undergone initial distillation was adjusted to an active ingredient content of 50% and DI water content of 50% by addition of DI water with thorough mixing at 60° C. Apart from the samples taken, the yield of dissolved N,N-dimethylglucamine was virtually quantitative.

The product obtained was characterized as follows:

| Appearance | clear, liquid, brown | |
|---|---|---|
| Gardner color number: | 8 | |
| Hazen color number: | >1000 | |
| Total amine number (titration): | 3.53 | meq/g |
| Solids content (105° C./2 hours): | 50.5% | |
| Water (Karl-Fischer titration): | 49.8% | (m/m) |
| N-Methylglucamine (GC): | 0.9% | (m/m) |
| N,N-Dimethylglucamine (GC): | 42.1% | (m/m) |
| Sorbitol (GC): | 0.8% | (m/m) |
| Methanol (GC): | <0.01% | (m/m) |
| Formaldehyde (free and chemically | 0.033% | (m/m) |
| bonded, LC): | | |
| Nitrosamines (total NNO): | <50 | μg/kg |
| Nickel (ICP-OES): | 45 | μg/g |
| Formic acid (free and chemically | 0.13% | (m/m) |
| bonded) (ion chromatography after | | |
| alkaline hydrolysis, IC) | | |

### Example 5

A nitrogen-inertized 50 liter stirred autoclave, equipped with temperature measurement, pressure measurement, safety valve, gas-introduction stirrer and immersed tube, was charged with 1.9 kg of water-moist Raney nickel (containing 40% water). The nitrogen atmosphere was then replaced by a hydrogen atmosphere. Subsequently 20.0 kg of 40.0% N-methylglucamine solution in water at 40° C. (Gardner color number: 1.4, Hazen color number: 202) were metered into the reactor. Following the leak test, the reactor stirrer was taken into operation, the internal temperature was adjusted to 40° C. and the hydrogen pressure to 85 bar. Over the course of 3 hours, 3.49 kg of aqueous formaldehyde solution (36.9% formaldehyde dissolved in water with max. 1.5% methanol) were metered continuously, using a pump, into the 50 liter stirrer autoclave. The molar ratio of N-methylglucamine to the total formaldehyde after the end of formaldehyde metering was 1.05. The stirring speed was held at 490 rpm and the internal temperature at 39-43° C. The hydrogen consumed was replaced by a supply of fresh hydrogen, and in this way the hydrogen pressure in the 50 liter stirred autoclave was held at 80-85 bar. After the end of hydrogen absorption, the after-reaction began. For this reaction, heating took place for 1 hour from 40° C. and 85 bar with stirring to 90° C., after which stirring was continued at 90° C. for 1, with cooling from 90° C. to 40° C. in 1 hour. After the end of the after-reaction, cooling took place to 30° C. and the stirrer was switched off. The Raney nickel settled for an hour. This was followed by a pressure filtration at 4 bar.

The filtered, aqueous N,N-dimethylglucamine was subjected to initial distillation in a batch process at 90° C. under a pressure of 600 mbar until the strength of the DMG solution was 50% (DMG-50). The distillate contained primarily water, a little methanol, and traces of other low boilers. Apart from the samples taken, the yield of dissolved N,N-dimethylglucamine was virtually quantitative.

The product obtained was characterized as follows:

| Appearance | clear, liquid, pale-yellow | |
|---|---|---|
| Gardner color number: | 0.5 | |
| Hazen color number: | 81 | |
| Total amine number (titration): | 132 | mg KOH/g |
| Solids content (105° C./2 hours): | 50.2% | |
| Water (Karl-Fischer titration): | 50.0% | (m/m) |
| N-Methylglucamine (GC) | 0.13% | (m/m) |
| N,N-Dimethylglucamine (GC): | 45.3% | (m/m) |
| Sorbitol (GC): | 0.7% | (m/m) |
| Methanol (GC): | <0.01% | (m/m) |
| Formaldehyde (free and chemically | 0.005% | (m/m) |
| bonded, LC): | | |
| Nitrosamines (total NNO): | <50 | μg/kg |
| Nickel (ICP-OES): | 0.6 | μg/g |
| Formic acid (free and chemically | 0.04% | (m/m) |
| bonded) (ion chromatography after | | |
| alkaline hydrolysis, IC) | | |

The influence of the hydrogenation pressure on the color number of the resulting product is evident from the inventive and comparative examples. A product with acceptable Hazen color number is obtained only in the pressure range from 10 to 200 bar hydrogen pressure.

1. A process for preparing an aqueous N,N-dimethylglucamine solution, wherein an aqueous solution of formaldehyde is metered into an aqueous solution of N-methylglucamine in the presence of a metal catalyst at a hydrogen pressure of 10-200 bar, in which subsequent to a hydrogenation with formaldehyde metering at 35-65° C., a further, after-hydrogenation step is added without formaldehyde metering at 70−110° C.

2. The process as claimed in claim 1, wherein the metal catalyst is Raney nickel.

3. The process as claimed in claim 1, in which the molar ratio of N-methylglucamine to formaldehyde is 1:1 to 1:1.5.

4. The process as claimed in claim 1, wherein the process is carried out at a hydrogen pressure of 20-180 bar.

5. The process as claimed in claim 1, in which the reaction temperature T is from 40-50° C.

6. The process as claimed in claim 1, wherein the Hazen color number of the resulting 50 wt % solution of N,N-dimethylglucamine in water is less than 800.

7. The process as claimed in claim 1, wherein the hydrogenation catalyst is used for more than 5 reaction batches.

8. The process as claimed in claim 1, wherein reaction takes place in a stirred reactor or loop reactor.

9. The process as claimed in claim 1, wherein the residual N-methylglucamine content is <2 wt %.

10. The process as claimed in claim 1, wherein the residual formaldehyde content is <0.1 wt %.

11. The process as claimed in claim 1, wherein subsequent to the hydrogenation a distillation step for removing methanol is appended.

12. The process as claimed in claim 1, in which the molar ratio of N-methylglucamine to formaldehyde is 1:1 to 1:1.2.

13. The process as claimed in claim 1, wherein the process is carried out at a hydrogen pressure of 70-120 bar.

14. The process as claimed in claim 1, wherein the Hazen color number of the resulting 50 wt % solution of N,N-dimethylglucamine in water is less than 400.

15. The process as claimed in claim 1, wherein the residual N-methylglucamine content is <1 wt %.

16. The process as claimed in claim 1, in which the molar ratio of N-methylglucamine to formaldehyde is 1:1.01 to 1:1.08.

17. The process as claimed in claim 1, wherein the Hazen color number of the resulting 50 wt % solution of N,N-dimethylglucamine in water is less than 230.

18. The process as claimed in claim 1, wherein the residual N-methylglucamine content is <0.25 wt %.
